(19) United States
(12) Patent Application Publication
(10) Pub. No.: US 2007/0043109 A1
(43) Pub. Date: Feb. 22, 2007

(54) Slimming cosmetic composition

(75) Inventors: Karl Linter, Rambouillet (FR); Claire Mas Chamberlin, Chevreuse (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Sederma Sas, Le Perray-en-Yvelines cedex (FR)

(21) Appl. No.: 10/557,616

(22) PCT Filed: May 19, 2004
(86) PCT No.: PCT/IB04/50755

§ 371 Date: Oct. 10, 2006

(30) Foreign Application Priority Data
May 20, 2003 (FR) 03/06063
Feb. 26, 2004 (JP) 2004-052321

(51) Int. Cl. A61K 31/343

(52) U.S. Cl. 514/468

(57) ABSTRACT

Cosmetic or dermopharmaceutical compositions containing, in a dermatologically acceptable carrier, at least one diterpene among: cafestol, kahweol and chemical derivatives are useful for slimming treatments including prevent and/or treat cellulite or orange peel and/or to refine contours of the face, skin firming, to reduce the steatomery of the thighs, to decrease the volume/fatty overload of the spare tire at the hips, to reduce “love handles”, both on men and women.

### CROSS-REFERENCE TO RELATED APPLICATIONS

The present application is a national phase entry under 35 U.S.C. §371 of International Application No. PCT/IB/2004/050755 filed May 19, 2004 published in English, which claims priority from French Application No. 03/06063 filed May 20, 2003, and Japanese Application No. 2004-052321 filed Feb. 26, 2004, all of which are incorporated herein by reference.

### TECHNICAL FIELD

The present invention relates to the field of cosmetics and personal care products.

### BACKGROUND OF THE INVENTION

Cosmetic industry permanently researches for new ingredients which possess real activities and which are usable in a topical manner by everyone.

The unsightliness of “spare tires” and localized fat deposits on thighs, long a preoccupation for women, has given rise to the development of numerous cosmetic slimming products. However, in order to accompany evolving attitudes in the overall management of localized fat overload problems, it is now necessary to address a wider public. In fact, while young and not so young women remain preoccupied by their figure, it is frequent for men to diet and practice body building targeted on the waist, hips and abdomen, zones that are particularly vulnerable to dietary relapses, a sedentary lifestyle and the cumulative effects of age.

Following the generation of slimming active substances based on direct activation of lipolysis via inhibition of phosphodiesterase (e.g. caffeine), more sophisticated products emerged. Those products address either stimulation of membrane receptors (G protein) or their inhibition (α-receptors and neuropeptide Y, NPY). Those approaches target activating glycerol release via adipocytic lipase (LHS). This approach induces depletion of cell lipid materials and hence a decrease in cell volume.

However, an innovative and complementary alternative route may exist: in addition to activating adipocytic fat store depletion, it is possible to deprive the cell of its supply source via the receptors enabling lipids to enter the cells. The adipocyte stores lipids supplied by the blood following ingestion of a meal using a special type of receptor: clathrin receptors.

Following a meal, cholesterol, triglycerides and other lipids are transported in the blood in the form of chylomicrons. Following partial hydrolysis, the chylomicrons yield various transport forms (see below), which constitute a system of addressing those forms to the various cell compartments (VALET and RICHARD Les lipides et la cellule adipeuse, NATHAN Editions, 1997).

For adipocytes, the address system mainly consists in LDL Low-density Lipoproteins) and VLDL Very Low-density Lipoproteins). We are now aware that adipocytes take in the nutrients required for maturation through the intermediary of LDL and VLDL. Triglyceride storage is largely related to the fatty acid released by VLDL (Yano et al., Atherosclerosis, 135 (1), p 57, 1997).

Following lipoprotein binding to clathrin receptors and endocytosis of the LDL receptor and VLDL receptor complexes, the triglyceride content and cholesterol transported are released in the adipocytes.

However, we discovered that the action of diterpenes like cafestol and kahweol as well as analogs and derivatives thereof on the regulation of the expression of the LDL and VLDL receptors allows to thwart lipidic storage of adipocytes while slowing down the development of neo-adipocytes.

### SUMMARY OF THE INVENTION

Surprisingly, it has now been found that diterpenes among cafestol, kahweol, as well as analogs and derivatives thereof, by topical application, have useful cosmetic activities as slimming agents, for a slimming treatment of weight overloads of the thighs and the hips, skin firming, and particularly prevent and/or treat cellulite or orange peel and/or to refine contours of the face.

It has been discovered that diterpenes chosen among cafestol, kahweol, as well as analogs and derivatives thereof, are new solutions as slimming agents both for men and women.

One goal of the present invention is to provide a cosmetic ingredient which offers an effectiveness as a slimming agent, and without notable side effect.

The present invention relates to a cosmetic or dermopharmaceutical slimming composition comprising, in a dermatologically acceptable carrier, at least one diterpene chosen among cafestol, kahweol, analogs and derivatives thereof.

In preferred embodiments, the present invention relates to cosmetic or dermopharmaceutical compositions that comprise at least one diterpene and at least one lipolytic agent other than the aforementioned diterpene in a dermatologically acceptable carrier. Preferred embodiments include an lipolytic agent selected among caffeine, theobromine, and/or methyl xanthines. Methods of using these cosmetics and dermopharmaeuticals for among others slimming, skin firming, preventing and/or treating orange peel or cellulite on both men an women are also contemplated.

### DETAILED DESCRIPTION

While the specification concludes with the claims particularly pointing and distinctly claiming the invention, it is believed that the present invention will be better understood from the following description.

All percentages and ratios used herein are by weight of the total composition and all measurements made are at 25° C., unless otherwise designated.

The compositions of the present invention can comprise, consist essentially of, or consist of, the components of the present invention as well as other ingredients described herein. As used herein, “consisting essentially of” means that the composition or component may include additional ingredients, but only if the additional ingredients do not materially alter the basic and novel characteristics of the claimed compositions or methods.

All publications cited herein are hereby incorporated by reference in their entirety.

The cosmetic compositions of the present invention contain at least one diterpene chosen among cafestol, kahweol, as well as analogs and derivatives of molecules having the Formula I. These are collectively referred to herein as “diterpenes” unless the context or the explicit statements indicate otherwise. One or more additional ingredients, dermatologically acceptable carrier(s), and/or adjuvants are also preferably used in these compositions.

The term “diterpenes” in accordance with the present invention is a compound of the following formula I: 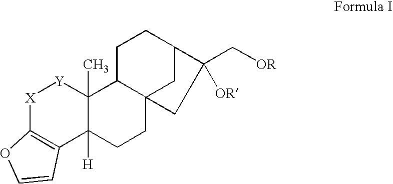
in which:
the groups R and R′, which groups may be the same or different, and each may be an hydrogen atom, an alkyl, aryl, aralkyl, acyl, sulfonyl or sugar group;
the X-Y sequence may be either CH₂—CH₂, CH═CH, CR¹R²—CR²R⁴ or CR⁵═CR⁶, in which the groups R¹, R², R³, R⁴, R⁵, R⁶, which groups may be the same or different, and each may be an hydrogen atom, an alkyl, aryl, aralkyl, acyl, sulfonyl or sugar group.

Particularly preferred diterpenes in accordance with the invention include cafestol (Formula I in which R═R′═H and X—Y═CH₂—CH₂) and kahweol (Formula I in which R═R′═H and X—Y═CH—CH). Also encompassed by the term diterpenes are substituted R groups. The topical compositions according to the present invention comprise at least one compound of the formula I.

The terms “alkyl” and “alkyl group,” in accordance with the present invention, mean any alkyl group of 1 to 20 carbon atoms linear or branched, substituted or unsubstituted (particularly with an alcohol, a carboxylic acid, an amine), saturated or insaturated. A particularly preferred alkyl group is the methyl group.

The terms “arayl” and “aryl group,” in accordance with the present invention, mean one or several aromatic cycles possessing each one from 5 to 8 carbon atoms, which can be joined or amalgamate, substituted or not substituted. Particularly, aryl groups can be phenyl groups, or naphtyl and substituents can be halogen atoms, alkoxy groups like described above, alkyl groups like described above or the nitro group.

The terms “aralkyl” and “aralkyl group,” in accordance with the present invention, mean any aryl group like abovedescribed, linked by an alkyl group like abovedescribed. A particularly preferred aralkyl group is the benzyl group.

The terms “acyl” and “acyl group,” in accordance with the present invention, mean any group —C═OR⁷ in which R⁷ can be either an alkyl group, an aryl group, an aralkyl group, an amine group, like abovedescribed. A particularly preferred acyl group is the acetyl group (R⁷═—CH₃)

The terms “amine” and “amine group,” in accordance with the present invention, mean any group —NR⁸R⁹ in which R⁸ and R⁹, which can be which can be identical or different, represent each one either an hydrogen atom, an alkyl group, an aryl group, an aralkyl group, an acyl group, a sulfonyl group, or a sugar group like abovedescribed.

The terms “sulfonyl” and “sulfonyl group,” in accordance with the present invention, mean any group —SO₂R¹⁰ in which R¹⁰ can be an alkyl group, an aryl group, an aralkyl group, an alkoxy group, an amine group, like abovedescribed. A particularly preferred sulfonyl group is the mesyl group (R¹⁰═—CH₃), triflyl group (R¹⁰═—CF₃), or tosyl group (R¹⁰═—Ph—CH₃).

The terms “alkoxy” and “alkoxy group,” in accordance with the present invention, mean any group —OR¹¹ in which R¹¹ can be, an alkyl group, an aryl group, an aralkyl group, an acyl group, a sulfonyl group, or a sugar group like abovedescribed.

The terms “sugar” and “sugar group,” in accordance with the present invention, mean any hexose group, ose group and oside group. Particularly preferred sugar groups are glucose groups, arabinose groups, fructose groups, galactose groups, mannose groups, maltose groups, lactose groups, saccharose groups, cellobiose groups.

Compounds in accordance with the present invention can possess one or more asymmetric centers and can exist thus then as individual optical isomers. The present invention relates to both the individual optical isomers and as mixtures thereof.

Particularly preferred compounds in accordance with the present invention are cafestol (formula I in which R═R′═H, X—Y═CH₂—CH₂), kahweol (formula I in which R═R′═H, X—Y═CH═CH), and cafestol acetate (formula I in which R═—COCH₃, R′═H, X—Y═CH₂—CH₂).

Diterpenes like cafestol, kahweol, and analogs and derivatives thereof can be obtained either by chemical synthesis, or by plant extraction, from any plant. A particularly preferred plant is coffee, and in particular green coffee bean, coffea Arabica (Coffee) Seed and coffea arabica bean. The extraction can be made according to a traditional extraction protocol.

Extraction solvents can be chosen among water, propylene glycol, butylene glycol, glycerin, PEG-6 Caprylic/capric glycerides, polyethylene glycol, methylic and/or ethylic diglycol ethers, cyclic polyols, ethoxylated or propoxylated diglycols, alcohols (methanol, ethanol, propanol, butanol), or any mixture of those solvents.

In addition, it is possible to carry out extracts of coffee by other processes as, for example, steeping, simple decoction, lixiviation, extraction under reflux, supercritical extraction, extraction with ultrasounds or micro-waves or finally with countercurrent technology, without this list being restrictive.

The amount of diterpene used according to the invention depends on the effect sought and must be a safe and effective amount to provide slimming. For example, the diterpenes are preferably used in cosmetic or dermopharmaceutical compositions as per the invention at concentrations which may range from about 0.1 ppm (0.00001% w/w also referred to herein as “weight percent,” “weight %” or simply by weight) to about 500,000 ppm (50% w/w), preferably from about 0.0001% w/w (1 ppm) to 10% w/w, and most preferably from about 0.001% w/w (10 ppm) to about 1% by weight of the composition.

In one preferred embodiments of the present invention, there is provided a personal care slimming product, a topical slimming composition, a cosmetic or dermopharmaceutical slimming composition (collectively a cosmetic slimming composition) which comprises at least a diterpene chosen among: cafestol, kahweol and analogs and derivatives thereof in a dermatologically acceptable carrier and optionally at least one lipolytic agent and/or an agent inhibiting lipogenesis, other than the aforementioned diterpene.

In order to complete the “denutrition” of adipocytes, it is, possible to add a complementarity of effect by stimulating release of the triglycerides already resident in the cell.

The usual pathway of amplification of cAMP via inhibition of phosphodiesterase is the first intention pathway. Phosphodiesterase inhibitors include xanthic bases and natural plant extracts containing them. Xanthic bases are, for example, caffeine, analogs and derivatives thereof, theophylline, analogs and derivatives thereof, theobromine, aminophylline, or xanthine, analogs and derivatives thereof such as methyl xanthines. Those xanthic bases can be used alone or in association/mixtures. The natural plant extracts containing xanthic bases of the present invention, include tea extracts, coffee extracts, guarana extracts, mate extracts and cola extracts.

Other examples of lipolytic agents which may be used according to the present invention are some plant extracts which do not contain caffeine, and some marine extracts.

Besides phosphodiesterase inhibitors, the lipolytic agent may be chosen among: α-2 blockers compounds capable of blocking α-2 receptors at the adipocytes surface, β-adrenergical agonists and antagonists, compounds stimulating β receptors and/or G proteins, glucose transport blockers, neuropeptide Y (NPY) antagonists capable of blocking NPY receptors at the adipocytes surface, agents modifying fat acids transport, lipolytic peptides and lipolytic proteins.

A preferred embodiment according to the present invention is the association of at least a diterpene chosen among: cafestol, kahweol, and analogs and derivatives thereof, with at least a methyl xanthine chosen among theobromine and caffeine, analogs and derivatives thereof.

Methyl xanthines like abovedescribed can be obtained either by chemical synthesis, by plant extraction, from any plant including them and particularly from Yerba mate. In one preferred aspect of the present invention, at least a diterpene chosen among: cafestol, kahweol, and analogs and derivatives thereof, is combined with an extract of Yerba mate, a plant of the high Andean plateaus which is the source of a popular stimulant beverage rich in methyl xanthines. Among them, caffeine and theobromine are the most represented and their effect on stimulation of triglyceride release was investigated.

Extraction solvents can de selected among water, propylene glycol, butylene glycol, glycerin, PEG-6 Caprylic/capric glycerides, polyethylene glycol, methylic and/or ethylic diglycol ethers, cyclic polyols, ethoxylated or propoxylated diglycols, alcohols (methanol, ethanol, propanol, butanol), or any mixture of those solvents.

In addition, it is possible to obtain extracts of Yerba Mate by other methods as, for example, steeping, simple decoction, lixiviation, extraction under reflux, supercritical extraction, extraction with ultrasounds or micro-waves or finally with countercurrent technology, without this list being restrictive.

Particularly preferred embodiments of the invention, include methyl xanthines from an extract of Yerba Mate titrated in caffeine and theobromine.

The amount of lipolytic or lipogenese inhibitor active ingredient, others than diterpene, present in the composition, can vary on a big range, and will be particularly from about 0.0001% w/w to about 50% w/w, preferably from about 0.001% w/w to 10% w/w, and most preferably from about 0.01% w/w to about 1% by weight of the composition.

One preferred embodiment of the present invention is the association of an extract of green coffee beans/seeds rich in diterpenes (particularly cafestol and kahweol) and of an extract of Yerba Mate rich in methyl xanthines, and particularly caffeine, theobromine, aminophylline, theophylline, xanthine. This association provides a complementarity of effect which leads to a global reduction of fatty mass by a reduction of the adipocytes number and volume, by the blockage of LDL and VLDL internalisation by adipocytes and by stimulation of the release of the triglycerides already resident in the cell.

It has now been found that such an association of active ingredients in a composition, comprising at least a diterpene chosen among: cafestol, kahweol and other analogs and derivatives thereof including those of Formula I discussed herein in a dermatologically acceptable carrier and, in a preferred embodiment, at least one lipolytic agent and/or an agent inhibiting lipogenesis, other than the aforementioned diterpene can be synergistic. Particularly, the use in a composition of the invention of the combination of at least a diterpene chosen among: cafestol, kahweol, and analogs and derivatives thereof, with at least a methyl xanthine chosen among theobromine and caffeine, analogs and derivatives thereof, may show a synergy of slimming effect. And more particularly, the use in a composition of the invention of the combination of an extract of green coffee beans/seeds rich in diterpenes (particularly cafestol and kahweol) and of an extract of Yerba Mate rich in methyl xanthines, and particularly caffeine, theobromine, aminophylline, theophylline, xanthine shows a synergy in slimming effect.

According to a preferred embodiment of the invention, the slimming composition includes, in addition to the diterpene, at least a complementary active ingredient selected from the group comprising:
Actives acting on micro-circulation (vasculoprotectors or vasodilatators) like flavonoids, Gingko biloba extracts, ruscogenines, naturals or synthetic esculosides, escine extracted from horse chestnut, nicotinates, hesperidine methyl chalcone, the ruscus or butcher's-broom, lavender or rosemary essential oils, extracts of Ammi visnaga;
Firming actives and/or anti-glycation actives (preventing sugar fixation on collagen fibers) as Centella asiatica extracts, Siegesbeckia extracts, silicium, amadorine, ergothioneine and derivatives, hydroxystilbenes and derivatives, for example resveratrol, plant extracts of the Ericaceae family, for exemple extracts of whortleberry, vitamine C and derivatives, retinol and derivatives, protein derivatives such as Integrissyme® (offered by SEDERMA SAS France) or fermentation products such as Kombuchka®, also offered by SEDERMA;
anti-cellulite agents. Suitable agents may include, but are not limited to, xanthine compounds (e.g., caffeine, theophylline, theobromine, and aminophylline). Especially useful are combinations with the cellulite/slimming agents called Vexel (FR 2 654 619 of Jan. 31, 1992), Coaxel (FR 2 694 195 of Jul. 30, 1992), Cyclolipase (FR 2 733 149 of Apr. 21, 1995), Pleurimincyl and Lipocare (WO 98/43607 of Oct. 8, 1998) all offered by SEDERMA.

The amount of these complementary active ingredients may vary on a large scale. When present in the compositions of the present invention, the composition preferably contains from about 0.0016 to about 50% w/w, more preferably from about 0.01 to 20% w/w, even more preferably from about 0.05% to about 10% w/w, still more preferably from 0.1% to about 5% by weight of the composition of the complementary active.

In one embodiment of the present invention, the slimming composition may contain, at least one diterpene, an extract of Yerba Mate, and/or an extract of green coffee bean and/or a Coffea Arabica (Coffee) Seed extract.

Other active ingredients may be advantageously used within the context of the present invention Specifically, the combination of the diterpenes that constitute the subject of the present invention with other cosmetic active substances (vide infra) is an advantageous implementation of the invention.

The term “dermatologically acceptable,” as used herein, means that the compositions or components described are suitable for use in contact with human skin without risk of toxicity, incompatibility, instability, allergic response, and the like.

All terms such as “slimming,” “skin firming,” “topical application,” and the like are used in the sense in which they are generally and widely used in the art of developing, testing and marketing cosmetic and personal care products.

The present invention intention includes, but is not limited to, prevention and/or reduction of all outward visibly and tactilely perceptible manifestations as well as any other macro or micro symptoms noticed on the skin due to excess weight. As used herein, therapeutically regulating excess weight by a topical application of the topical composition of the present invention includes inducing a slimming treatment of weight overloads of the thighs and the hips, skin firming, and particularly preventing and/or treating cellulite or orange peel and/or refining contours of the face. It also includes reduction of steatomery of the thighs, reduction of the volume/fatty overload of the spare tire at the hips, reduction of “love handles”. Note that all those processes may happen both on men and women. The present invention has been found to act again excess weight both on men and women.

The term “cosmetic or dermopharmaceutical composition” or “cosmetic composition” or “topical composition” or more briefly just “composition” in accordance with the present invention relates to a formulation that can be used for cosmetic purposes, purposes of hygiene or as a basis for delivery of one or more pharmaceutical ingredients. It is also possible that these formulations are used for two or more of these same purposes at one time. At a minimum, these compositions include a diterpene, analogs or derivatives thereof. These compositions may also include additional ingredients such as a dermatologically acceptable carrier.

“Cosmetics,” as used herein, include without limitation, lipstick, mascara, rouge, foundation, blush, eyeliner, lipliner, lip gloss, facial or body powder, sunscreens and blocks, nail polish, mousse, sprays, styling gels, nail conditioner, whether in the form of creams, lotions, gels, ointments, emulsions, colloids, solutions, suspensions, compacts, solids, pencils, spray-on formulations, brush-on formulations and the like. “Personal care products” include, without limitation, bath and shower gels, shampoos, conditioners, cream rinses, hair dyes and coloring products, leave-on conditioners, sunscreens and sunblocks, lip balms, skin conditioners, cold creams, moisturizers, hair sprays, soaps, body scrubs, exfoliants, astringents, depilatories and permanent waving solutions, antidandruff formulations, antisweat and antiperspirant compositions, shaving, preshaving and after shaving products, moisturizers, deodorants, cold creams, cleansers, skin gels, rinses, whether in solid, powder, liquid, cream, gel, ointment, lotion, emulsions, colloids, solutions, suspensions, or other form. “Pharmaceutical preparations” in accordance with the present invention include, without limitation, carriers for dermatological purposes, including topical and transdermal application of pharmaceutically active ingredients. These can be in the form of gels, patches, creams, nose sprays, ointments, lotions, emulsions, colloids, solutions, suspensions, powders and the like. Compositions in accordance with the invention include cosmetics, personal care products and pharmaceutical preparations. The compositions are, for example, emollient lotions, milks or creams, milks or creams for skin or hair care, makeup cleansing creams, lotions or milks, foundation bases, sunscreen lotions, sun milks or creams, artificial sun-tanning lotions, artificial sun-tanning milks or creams, shaving creams or foams, aftershave lotions, shampoos, lipsticks, mascaras or nail varnishes.

The diterpenes may be used in cosmetic compositions in accordance with the present invention either as individual additions or as a premix in a suitable carrier, and may be in the form of a solution, dispersion, emulsion paste or powder. They may be included individually or with other active substances, cited or not cited, in vehicles such as macrocapsules, microcapsules or nanocapsules, macrospheres, microspheres or nanospheres, liposomes, oleosomes or chylomicrons, macroparticles, microparticles or nanoparticles, macrosponges, microsponges or nanosponges. They may also be adsorbed on powdered organic polymers, talcs, bentonites and other inorganic carriers.

The diterpenes and the composition containing the same may be used in any form whatsoever or in a form bound, incorporated, absorbed in or adsorbed on macro-, micro- and nanoparticles, macro-, micro- and nanocapsules for the treatment of textiles, synthetic or natural fibers, wools and any materials that may be used for clothing or underwear for the day or night, intended for contact with the skin, such as pantyhose, underwear, handkerchiefs and wipes, active materials of any sort, wipes, patches, compresses, cottons, cotton buds, dressings, makeup-removal sponges, masks and any other carrier liable to come into direct contact with the skin or scalp to enable continuous topical delivery in order to exert a cosmetic effect through the contact between the textile and skin and/or scalp.

Some of the compositions of the present invention may also provide additional benefits, including stability, absence of significant (consumer-unacceptable) skin irritation, anti-inflammatory activity and good aesthetics.

When the compositions compliant with the invention have the form of water-in-oil or oil-in-water emulsions, the oily phase may include a mixture of an extracted or synthetic fatty substances with at least one oil and possibly another fatty substance. The oily phase of the emulsions may constitute 5 to 60% of the total weight of the emulsion.

The aqueous phase of the said emulsions preferably constitutes 30 to 85% of the total weight of the emulsion. The proportion of emulsifier may be between 1 and 20% and preferably between 2 and 12% of the total weight of the emulsion. When these composition according to the invention are in the form of oily, oil in alcohol or dilute alcohol lotions, the compositions may constitute, for example, sunscreen lotions containing a filter absorbing UV radiation or softening lotions for the skin. Oily lotions may also constitute foaming oils containing an oil-soluble surfactant, bath oils, etc.

Among the main adjuvants that may be present in the compositions according to the invention are organic or hydroglycolic solvents, including MP-diol and polyglycerols, extracted or synthetic fats, ionic or non-ionic thickeners, softeners, opacifiers, stabilizers, silicones, α- or β-hydroxyacids, vitamins, fragrances, preservatives, sequestering agents, colorants, gelling and viscosifying polymers, surfactants and emulsifiers, other water- or fat-soluble active substances, plant extracts, tissue extracts, marine extracts, sun filters and antioxidants, moisturizing agents, softening agents, products for the treatment of skin diseases, germicides, propellants. Adjuvants are used synonomously with “additional ingredients” and a further list of some is provided elsewhere.

The most preferred mono- or poly-alcohols are selected from ethanol, isopropanol, propylene glycol, glycerol and sorbitol. As the fatty substance, among the mineral oils, may be cited liquid paraffin, among the animal oils, whale, shark, seal, menhaden, haddock liver, cod, tuna, tortoise, calf's foot, horse's foot, sheep's foot, mink, otter and marmot oil, etc. Among the plant oils may be cited almond, wheat germ, jojoba, sesame, sunflower, palm, Brazil nut, shea, shorea, macadamia, blackcurrant seed and similar oils.

Among the fatty acid esters useful as a fatty substance may be used C₁₂ to C₂₂ acid esters, saturated or unsaturated, and lower alcohols such as isopropanol and glycerol, or C₈ to C₂₂ acids, linear or branched, saturated or unsaturated, or 1,2-alkanediols of C₁₀ to C₂₂.

Other fatty substances include liquid paraffin, paraffin, waxes, lanolin, hydrogenated lanoline, tallow, acetylated lanoline and silicone oils. Among the waxes may be cited Sipol wax, lanoline wax, beeswax, Candelila wax, monocrystalline wax, Carnauba wax, spermaceti, cocoa butter, shea butter, silicone waxes, hydrogenated oils solid at 25° C., sucroglycerides, oleates, myristates, linoleates and calcium, magnesium and aluminum stearates.

Among the fatty alcohols may be cited lauryl alcohol cetyl alcohol, myristyl alcohol, stearyl alcohol, palmityl alcohol, oleyl alcohol and Guerbet alcohols such as 2-decyltetradecanol or 2-hexyldecanol. As emulsifiers, among the polyoxyethylenated fatty alcohols may be cited lauryl, cetyl, stearyl and oleyl alcohols, containing 2 to 20 moles of ethylene oxide, alcohols. Among the alkyl ethers may be cited glycerol and C₁₂-C₁₈ alcohols with 2 to 10 moles of glycerol. Thickeners may also be of value, such as cellulose derivatives, polyacrylic acid derivatives, guar gum, carouba and xanthan gum.

When the compositions according to the invention are dispersions, they may consist of dispersions of lecithin in water in the presence of a surfactant or aqueous dispersions of lipid spherules consisting of organized molecular layers enclosing an encapsulated aqueous phase. In that context, as lipid compounds, may be cited long-chain alcohols and diols, sterols such as cholesterol, phospholipids, cholesteryl sulfate and phosphate, long-chain amines and quaternary ammonium derivatives, dihydroxyalkylamines, polyoxyethylenated fatty amines, long-chain aminoalcohol esters, their salts and quaternary ammonium derivatives, phosphoric esters of fatty alcohols such as acid dicetylphosphate or its sodium salt, alkylsulfates such as sodium cetylsulfate, fatty acids in the form. of salts or lipids of the type described in French patent No. 2 315 991, No. 1 477 048 and No. 2 091 516 or in international patent applications WO 83/01 571 and WO 92/08685.

As examples of other lipids that may be used may be cited lipids carrying a long lipophilic chain of 12 to 30 carbon atoms, saturated or unsaturated, branched or linear, for example, an oleic, lanolic, tetradecylic, hexadecylic, isostearylic, lauric or alkylphenylic chain. The hydrophilic group of those lipids may be an ionic or non-ionic group. As examples of non-ionic groups may be cited those derived from polyethylene glycol. As lipids forming the lamellar phase, the following may advantageously be used: polyglycerol ethers such as those described in French patents No. 1 477 048 , No. 2 091 516, No. 2 465 780 and No. 2 482 128.

As an ionic group, a group derived from an amphoteric, anionic or cationic compound may advantageously be used.

Other lipids described in the international patent application, WO 83/01 571, as suitable for use in the formation of vesicles are glycolipids such as lactosylceramide, galactocerebroside, gangliosides and trihexosylceramide together with phospholipids such as phosphatidyl glycerol and phosphatidyl inositol.

Additional Ingredients

In addition to the diterpene, analogs and/or derivatives thereof described herein, the composition of the invention may include various other and additional ingredients, which may be active, functional, conventionally used in cosmetic, personal care or topical/transdermal pharmaceutical products or otherwise, which provide some benefit to the object of the composition. Such additional ingredients may include one or more substances such as, without limitations, cleaning agents, hair conditioning agents, skin conditioning agents, hair styling agents, antidandruff agents, hair growth promoters, perfumes, sunscreen and/or sunblock compounds for hair and/or skin, detergents, pharmaceuticals, humectants, emollients, antiseptic agents, deodorant actives.

The compositions of the present invention generally contain at least one additional ingredient. The compositions of the present invention may contain a plurality of additional ingredients as well. Usually these compositions include at least one dermatologically acceptable carrier.

In a preferred embodiment, where the composition is to be in contact with human keratinous tissue, the additional ingredients should be suitable for application to keratinous tissue, that is, when incorporated into the composition they are suitable for use in contact with human keratinous tissue (hair, nails, skin, lips) without undue toxicity, incompatibility, instability, allergic response, and the like within the scope of sound medical judgment. The CTFA Cosmetic Ingredient Handbook, Ninth Edition (2002) describes a wide variety of nonlimiting cosmetic and pharmaceutical ingredients commonly used in the skin care industry, which are suitable for use as additional ingredients in the compositions of the present invention. Non-limiting examples of these additional ingredient classes include: abrasives, absorbents, aesthetic components such as fragrances, pigments, colorings/colorants for hair and skin, make-up agents, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), binders, biological additives, buffering agents, bulking agents, chelating agents, chemical additives, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, thickeners, peptides and peptide derivatives thereof, vitamins and derivatives thereof.

The active substances which may be combined with the diterpenes, may, in addition to those previously discussed, be substances of pharmaceutical or dietary value or be endowed with their own cosmetic activity. When those active substances are water-soluble, they may be uniformly dissolved or they may be dissolved in an encapsulated aqueous phase inside vesicles. The water-soluble substances with cosmetic and/or pharmaceutical activity may be products intended for care or treatment of the skin and hair, such as, for example, wetting agents like glycerol, sorbitol, pentaerythritol, pyrrolidone acid and its salts; artificial sun-tanning agents such as dihydroxyacetone, erythrulose, glyceraldehyde, and γ-dialdehydes such as tartaraldehyde, those compounds possibly being combined with colorants; water-soluble sunscreens; antiperspirants, deodorants, astringents, and refreshing, tonic, cicatrizing, keratolytic and depilatory products, perfumed water; botanical extracts, plant tissue extracts such as polysaccharides; water-soluble colorants; anti-dandruff agents; antiseborrheic agents, oxidants such as bleaching agents such as hydrogen peroxide; reducing agents such as thioglycolic acid and its salts.

The following may also be cited: vitamins B₁ through B₁₂, C, D, H, K and their derivatives, peptide or steroid hormones, enzymes (e.g., superoxide dismutase), coenzymes, enzyme inhibitors, enzyme activators, vaccines, steroidal or non-steroidal anti-inflammatories such as hydrocortisone, antibiotics, antimicrobial and bactericidal substances, cytotoxic or antineoplastic agents.

When the active substances are fat soluble, they may be incorporated in the lipid phase of the emulsions or in the membranes of vesicles such as liposomes, micelles and chylomicrons. The active substances may be selected in the group formed by fat-soluble active substances selected from the group formed by the fat-soluble sunscreens, substances intended to improve the state of dry or aged skin, tocopherols, vitamins E, F or A and their esters, retinoic acid, antioxidants, essential fatty acids, glycyrrhetinic acid, keratolytics and carotenoids, ceramides and pseudo-ceramides, and all lipid complexes of a form similar to that of the natural ceramides of the skin.

The present invention also relates to a method comprising a topical application of a cosmetic or dermopharmaceutical composition according to the invention, of preventing and/or reducing all outward visibly and tactilely perceptible manifestations as well as any other macro or micro symptoms noticed on the skin due to excess weight, of therapeutically regulating excess weight, of inducing a slimming treatment of weight overloads of the thighs and the hips, skin firming, and particularly of preventing and/or treating cellulite or orange peel and/or refining contours of the face, of reducing the steatomery of the thighs, the volume/fatty overload of the spare tire at the hips, of reducing “love handles,” on men and women skin which need such treatment.

The present invention also relates to the use of a composition according to the invention, like or for the manufacturing of a cosmetic or dermopharmaceutical composition for a slimming treatment of weight overloads of the thighs and the hips, skin firming, and particularly prevent and/or treat cellulite or orange peel and/or to refine contours of the face.

The present invention concerns a cosmetic treatment in order to prevent and/or treat cellulite and/or orange peel and/or refine contours of the face, comprising a topical application of a cosmetic or dermopharmaceutical composition according to the invention.

The composition of the invention can be locally applied on the parts of the face or of the body which need such a treatment, particularly on hips, buttocks, thighs, the belly, and the oval of the face. One of the advantages of the present invention is in the possibility of being available to proceed, each time it is necessary or desirable, to “soft” treatments very localized and selective thanks to the application way by topical route.

The present invention concerns also the use of a composition of the invention to make cosmetics, personal care products, topical pharmaceutical preparations or medicines intended for skin, mucous membrane, hair and nail care, particularly intended for a slimming treatment of excess weight of the thighs and the hips, skin firming, and particularly prevent and/or treat cellulite or orange peel and/or to refine contours of the face.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. As an illustration of the invention, several cosmetic formulae will be cited. The formulae are representative of, but do not restrict, the invention:

### Example 1

### Slimming Preparation (SP1)

| Ingredients | g/100 g |
|---|---|
| Yerba Mate extract | 30.0 |
| Butylene glycol | 15.0 |
| Green coffee beans extract | 10.0 |
| Crovol A70 | 5.0 |
| Glycerin | 5.0 |
| Cetyl hydroxyethylcellulose | 1.5 |
| Water, preservatives | qsp 100 g |

This preparation SP1 guarantees the following active quantities:

| Active ingredient | % w/w of SP1 |
|---|---|
| Cafestol | 0.05-0.07% |
| Caffeine | 0.9-1% |
| Theobromine | 0.09-0.11% |

This preparation, SP1, is one preferred form of the invention. The amount of this preparation in a cosmetic composition may vary in a large scale and will be preferably between around 0.001% and around 20% w/w, more preferably between around 0.01% and around 10% w/w, by the total weight of the cosmetic composition. Preparation SP1 may be the slimming active ingredient of the following examples.

### Example 2

### Slimming Gel

| Ingredients | g/100 g |
|---|---|
| Carbopol 1342 | 0.3 |
| Propylene glycol | 2.0 |
| Glycerin | 1.0 |
| White paraffine | 1.5 |
| Cylomethicone | 6.0 |
| Cetylic alcohol | 0.5 |
| Lubrajel MS | 10.0 |
| Triethanolamine | 0.3 |
| SP1 | 3.0 |
| Water, preservatives, fragrance | qsp 100 g |

### Example 3

### Massage Slimming Cream

| Ingredients | g/100 g |
|---|---|
| Ultrez 10 | 0.2 |
| Butylene glycol | 5.0 |
| Stearic acid | 1.5 |
| Crodamol GTCC | 2.0 |
| Petrolatum oil | 2.0 |
| Crodacol C90 | 0.5 |
| Crodafos CES | 1.5 |
| SP1 | 3.0 |
| Water, preservatives, fragrance | qsp 100 g |

### Example 4

### In Vitro Studies: Effect of Cafestol Acetate on the mRNA of LDLr and VLDLr Receptors

To evidence regulation of transcription (control of the number of copies of the messenger RNA for the gene coding for the protein studied), it was necessary to isolate the messenger RNA (mRNA) synthesized from the cell culture. The mRNA specific for the protein of interest must be recognized in the total mRNA pool present in the cell.

The used method is RT-PCR, of amplification and quantization, conducted in real time on the latest-generation systems and which enables in-line quantitative measurement of the target mRNA.

Protocol

Neo-adipocytes 3T3-L1 in the adipocytic differentiation phase (after addition of an appropriate triggering cocktail) were incubated with cafestol acetate for 24 hours.

At the end of the test, the adipocytes were lysed and the extracted mRNA transcribed to cDNA (by reverse transcriptase). The cDNA were then amplified (n copies using Taq polymerase) to obtain a detectable signal.

The amplicons (cDNA amplification products) were quantified directly, as they emerged in the RT-PCR system, then subjected to electrophoresis in order to visually illustrate the results.

Results

TABLE 1
Quantification of mRNA expression in adipocytes incubated with
cafestol acetate for 24 hours. Values standardized on the internal
standard, β-actin, were then expressed as percentages relative
to the control. Mean results for n = 3 tests

| | mRNA expression: Values standardized | |
|---|---|---|
| | on the internal standard | |
| | VLDL receptor | LDL receptor |
| Control | 100 | 100 |
| Cafestol Acetate 15 ppm | 90 | 66 |

Cafestol acetate showed a very marked effect, decreasing mRNA expression of the LDL receptor. The reduction was −33%, equivalent to that of the positive control, and 10% of reduction of the VLDLr in 24 hours.

### Example 5

### In Vivo Studies

BODY CREAM formula used for the clinical study on the female panel:

| Ingredients | g/100 g |
|---|---|
| Ultrez 10 | 0.4 |
| Glycerin | 10.0 |
| Crillet 1 | 2.0 |
| Crodamol OP | 4.0 |
| Dimethicone | 3.0 |
| Potassium sorbate | 0.1 |
| NaOH 38% | 0.6 |
| Ethanol | 3.0 |
| SP1 | 3.0 |
| Water, preservatives, fragrance | qsp 100 g |

MASSAGE CREAM formula used for the clinical study on the male panel:

| Ingredients | g/100 g |
|---|---|
| Ultrez 10 | 0.2 |
| Buthylene glycol | 5.0 |
| Crodamol GTCC | 2.0 |
| Crodamol C90 | 0.5 |
| Dimethicone | 1.5 |
| DC345 | 2.0 |
| Potassium sorbate | 0.1 |
| Stearic acid | 1.5 |
| NaOH 38% | 0.5 |
| Ethanol | 3.0 |
| SP1 | 3.0 |
| Water, preservatives, fragrance | qsp 100 g |

The in vivo efficacy study on SP1 was conducted using a female panel and a male panel. The method known as interference-fringe topometry was used. The method consists in analysis of the optical deformation of fringe projection under laser illumination (Fast Optical In vivo Topometry of human Skin, FOITS).

Principle

The subjects are placed at a precise distance and in a position that was perfectly reproducible from one measurement to the next. The zones under analysis are successively scanned by fringes of different widths. The measurements are conducted on a site precisely located in terms of natural features such as beauty spots and visible venules.

The deformation related to the state of the surface scanned is automatically recorded for increasingly narrow projected fringes. The automatic signal acquisition using a CCD camera enables reconstitution of the volume explored without direct contact with the subject. The volume explored is expressed in mL and the changes in volume between the start and end of the study enable quantitation of the local loss.

Protocol

The volunteers, 15 male subjects and 12 female subjects, applied the cream twice daily for 56 days. Each subject acted as his/her own control and the treated side was compared with the untreated contralateral side. So doing eliminated any contextual variation in volume independent of the effect studied during the study.

Study on the Female Panel

Inclusion Criteria:

Women aged 18 to 45 years, Caucasian, with steatomery of the thighs, BMI between 20 and 26 kg/m², weight stable for at least 3 months, no slimming diet in the 3 months preceding the study.

Results:

The 3D acquisitions enabled reconstruction of thigh volume between 2 inferior and superior levels. Twelve volunteers of mean age 28.7±8 years took part in the study. Thigh volume was compared at T0 and T56. The thigh perimeter parameter is given for the superior transverse section, the upper volume cutoff. At T0, there was no significant difference between the volumes of the right and left thighs. The mean changes in volume after 56 days were determined and the significance tested using Student's t test for paired series (treatment vs. control) and an unpaired t test (difference: T0 vs. T56 for each thigh).

TABLE 2
Mean change in thigh volume, T0 vs. T56 days, after twice
daily application of 3% SP1 by a panel of 12 women

| | Thigh volume after 56 days of | | | |
|---|---|---|---|---|
| | 3% SP1 treatment | | | |
| | Treated | | Untreated | |
| FEMALE PANEL | T0 | T56 | T0 | T56 |
| Volume (mL) | 2662 | 2391 | 2512 | 2564 |
| % change | — | −10.07% | — | +0.2% |

| | | |
|---|---|---|
| Maximum % change | −27% | — |
| Significance/T0 | p < 0.01 | NS |

| | |
|---|---|
| Significance vs. untreated | p < 0.05 |

TABLE 3
Mean change in thigh perimeter, T0 vs. T56 days, after twice
daily application of 3% SP1 by a panel of 12 women.

| | Thigh perimeter after 56 days of | | | |
|---|---|---|---|---|
| | 3% SP1 treatment | | | |
| | Treated | | Untreated | |
| FEMALE PANEL | T0 | T56 | T0 | T56 |
| Perimeter (cm) | 57.27 | 56.50 | 57.29 | 57.38 |

| | | |
|---|---|---|
| Change | −0.8 cm | +0.1 cm |
| Maximum change | 1.9 cm | — |
| Significance/T0 | p < 0.01 | NS |

| | |
|---|---|
| Significance vs. untreated | p < 0.05 |

After 56 days of application, thigh volume and perimeter showed very significant changes with a 10% decrease in volume (p<0.01) and a reduction in thigh perimeter of nearly 1 cm (p<0.01). Over the same period, there was no significant difference for the untreated thighs and even a non-significant very slight increase. Daily use of 3% SP1 thus demonstrated its ability to reduce fat overload of the thighs in women.

Study on the Male Panel

Inclusion Criteria

Subjects aged ≧30 years, Caucasian, BMI between 20 and 26 kg/m², presence of fat overloads in the form of visible spare tire at the hips, weight stable for at least 3 months, no slimming diet in the 3 months preceding the study.

Results

The 3-D acquisitions enabled reconstruction of the lateral surface and volume of the spare tire on the hips. In this case, the accessible parameter was a fictitious volume (since, in contrast to what was done with the thigh, only the lateral contour can be explored) taking into account the mean distance separating the surface of the spare tire and a fictional section plane (describing a section of the-thorax) that was precisely positioned. Accordingly, for each subject, the mean variation in the amplitude of the spare tire was determined in mL and could not be converted to a percentage.

Fifteen volunteers of mean age 42.3±11 years took part in the study. At T0, there was no significant difference between the volume of the spare tire on the right and left. The mean changes in volume of the projecting part of the hip after 56 days were determined and the significance calculated using Student's t test for paired series (treatment vs. control) and unpaired series (difference, T0 vs. T56, for each hip).

TABLE 4
Mean changes in spare tire amplitude, T0 vs. T56 days, after
twice daily application of 3% SP1 by 15 male volunteers.

Spare tire amplitude after 56 days of
treatment with 3% SP1

| MALE PANEL | Treated side | Untreated side |
|---|---|---|
| Volume change (mL) | −17.7 | −4.2 |
| Significance/T0 | p < 0.059 | NS |

After 56 days of application, the volume of the spare tire at the hip decreased by 17.7 mL. Over the same period, there was no significant difference for the untreated hip. Used daily, 3% SP1 demonstrated its ability to reduce fatty overload of the hips (spare tire) in men.

### Example 6

### Slimming Anti-stretchmark Gel

| Ingredients | | % by wt. |
|---|---|---|
| Part A | | |
| Water Deionised | — | qs 100 |
| Part B | | |
| Butylene Glycol | — | 5.00 |
| Phenova [Phenoxyethanol (and) | Crodarom | 0.80 |
| Mixed Parabens] | | |
| Part C | | |
| Crill 3 [Sorbitan Stearate] | Croda | 1.20 |
| Crillet 3 [Polysorbate 60] | Croda | 3.00 |
| DC 200 [Dimethicone] | Dow Corning | 2.00 |
| Crodamol IPM [Isopropyl Myristate] | Croda | 5.00 |
| Crodamol W [Stearyl Heptanoate] | Croda | 0.30 |
| Crodamol GTCC [Caprylic/Capric | Croda | 5.00 |
| Triglyceride] | | |
| Crodacol CS90 [Cetearyl Alcohol] | Croda | 2.00 |
| Ceramide 2 [N-stearoylsphinganine] | SEDERMA | 0.10 |
| Part D | | |
| Carbopol 980 at 2% [Carbomer] | BF Goodrich | 10.00 |
| Part E | | |
| Potassium Sorbate | — | 0.10 |
| Part F | | |
| Water Deionised | — | 2.00 |
| Sodium Hydroxide | — | 0.20 |
| Part G | | |
| Water | | 10.0 |
| DERMAXYL ® | SEDERMA | 3.00 |
| Rutin | | 0.1 |
| Bowman Birk Inhibitor | | 0.0001 |
| UNISLIM ® | SEDERMA | 3.00 |

UNISLIM®: Ilex Paraguariensis (Leaf) Extract (and) Water (and) Butylene Glycol (and) Coffea Arabica (Coffee) Seed Extract (and) PEG-60 Almond Glycerides (and) Glycerin (and) Cetyl Hydroxyethylcellulose.

DERMAXYL®: C12-15 Alkyl Benzoate(and) Tribehenin (and) Ceramide 2 (and) PEG-10 Rapeseed Sterol (and) Palmitoyl Oligopeptide

This gel can be prepared in the following way: Homogenize Part B and pour it into Part A. Heat Part (A+B) to 75° C. Heat Part C and Part D to 75° C. Pour Part C into Part (A+B) with helix stirring; then, pour Part D into Part (A+B+C). Add Part F and Part E. Pour Part G at about 35° C.

### Example 7

### Slimming Anti Stretch-Mark Cream

| Ingredients | | % by wt |
|---|---|---|
| Part A | | |
| Water Deionised | — | qs 100 |
| Ultrez 10 [Carbomer] | Noveon | 0.40 |
| Part B | | |
| Glycerin | Croda | 5.00 |
| Phenova [Phenoxyethanol (and) | Crodarom | 0.80 |
| Mixed Parabens] | | |
| Part C | | |
| Crodamol OP [Ethylhexyl Palmitate] | Croda | 4.00 |
| Crodacol CS90 [Cetearyl alcohol] | Croda | 0.50 |
| Crodamol ML [Myristyl Lactate] | Croda | 0.30 |
| Crillet 1 [Polysorbate 20] | Croda | 1.00 |
| Part D | | |
| Pemulen TR2 [Acrylates/C 10-30 Alkyl | Noveon | 0.20 |
| Acrylate (and) Crosspolymer] | | |
| DC 345 [Cyclomethicone] | Dow Corning | 2.00 |
| Part E | | |
| Potassium Sorbate | — | 0.10 |
| Part F | | |
| Water | — | 6.00 |
| Sodium Hydroxide 38% | — | 0.60 |
| Part G | | |
| MATRIXYL ® 3000 | SEDERMA | 3.00 |
| Darutoside (Siegesbeckia Orientalis Extract) | SEDERMA | 2.00 |
| UNISLIM ® | SEDERMA | 2.00 |

Darutoside is a molecule sold by SEDERMA for the treatment of stretch marks.

MATRIXYL® 3000 is sold by SEDERMA for the treatment of wrinkles [Glycerin (and) Water (Aqua) (and) Butylene Glycol (and) Carbomer (and) Polysorbate 20 (and) Palmitoyl Oligopeptide (and) Palmitoyl Tetrapeptide-3].

UNISLIM®: Ilex Paraguariensis (Leaf) Extract (and) Water (and) Butylene Glycol (and) Coffea Arabica (Coffee) Seed Extract (and) PEG-60 Almond Glycerides (and) Glycerin (and) Cetyl Hydroxyethylcellulose.

This emulsion is prepared in the following way: Phase A: disperse Ultrez 10 in water and let it swell for 20 minutes, then add phase B; heat to 75 C°. Heat Phase C separately to 75° C. Mix the two phases under stirring, homogenise, add Phase D, neutralise with Phase E, cool until reaching 30° C., then add Phase F and Phase G, adjust pH to ˜6 with NaOH.

### INDUSTRIAL APPLICABILITY

The present invention relates to the chemical, medical, cosmetic and personal care industries.

1-18. (canceled)

19. A cosmetic or dermopharmaceutical slimming composition comprising: in a dermatologically acceptable carrier, at least one diterpene corresponding
to the general formula I: 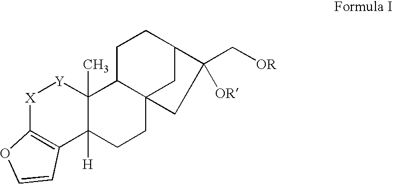
in which:
the groups R and R′, which groups may be the same or different, and each may be an hydrogen atom, an alkyl, aryl, aralkyl, acyl, sulfonyl or sugar group;
the X—Y sequence may be either CH₂—CH₂, CH═CH, CR¹R²—CR²R⁴ or CR⁵═CR⁶, in which the groups R¹, R², R³, R⁴, R⁵, R⁶, which groups may be the same or different, and each may be an hydrogen atom, an alkyl, aryl, aralkyl, acyl, sulfonyl or sugar group.

20. The topical composition of claim 19, wherein said diterpene is obtained from a plant extract.

21. The topical composition of claim 20, wherein said plant extract is a green coffee seed extract.

22. the topical composition of claim 19, wherein said diterpene is cafestol or kahweol.

23. The topical composition of claim 19, further comprising: at least one compound selected from lipolytic agents and agents inhibiting lipogenesis.

24. The topical composition of claim 23, wherein said lipolytic agent is a phosphodiesterase inhibitor, α-2 blocker, β-adrenergical agonist, β-adrenergical antagonist, compound stimulating β receptor, compound stimulating G protein, glucose transport blocker, neuropeptide Y (NPY) antagonist capable of blocking NPY receptors at the adipocytes surface, an agent modifying fat acid transport, lipolytic peptide or lipolytic protein.

25. The topical composition of claim 24, wherein said phosphodiesterase inhibitor is selected among xanthic bases and natural plant extracts containing them.

26. The topical composition of claim 25, wherein said xanthic bases are selected among the group comprising: caffeine, theobromine, aminophylline, theophylline, xanthine, analogs and derivatives thereof.

27. The topical composition of claim 25, wherein said xanthic bases are methyl xanthines.

28. The topical composition of claim 23, wherein said lipolytic agent inhibiting lipogenesis is caffeine and/or theobromine.

29. The topical composition of claim 19, wherein said diterpene is present in an amount of between about 0.00001% (w/w) and about 50% (w/w) by weight of the composition.

30. The topical composition of claim 29, wherein said diterpene is present in an amount of between about 0.0001% (w/w) and about 10% (w/w).

31. The topical composition of claim 30, wherein said diterpene is present in an amount of between about 0.001% (w/w) and about 1% (w/w).

32. The topical composition of claim 23, wherein said lipolytic agent or agent inhibiting lipogenesis is present in an amount of between about 0.0001% (w/w) and about 50% (w/w), by weight of the composition.

33. The topical composition of claim 32, wherein said lipolytic agent or agent inhibiting lipogenesis is present in an amount of between about 0.001% (w/w) and about 10% (w/w).

34. The topical composition of claim 33, wherein said lipolytic agent or agent inhibiting lipogenesis is present in an amount of between about 0.01%(w/w) and about 1% (w/w)

35. The topical composition of claim 25, wherein said natural plant extract is a Yerba Mate extract.

36. The topical composition of claim 19, further comprising at least a complementary active ingredient selected from the group comprising: actives acting on micro-circulation, firming actives and/or anti-glycation actives and their mixtures.

37. The topical composition of claim 19, wherein said diterpene is present in the form a solution, dispersion, emulsion, paste or powder, or is included individually or as a premix, in vehicles constituted by carriers such as macro-, micro- or nanocapsules, macro-, micro- or nanospheres, liposomes, oleosomes or chylomicrons, macro-, micro- or nanoparticles, macro-, micro- or nanosponges, or are adsorbed on organic polymers powders, talcs, bentonites or other inorganic carriers.

38. The topical composition of claim 19, which is an emollient lotion, milk, or cream, milk or cream for skin or hair care, makeup cleansing lotions or milks.

39. The topical composition of claim 19, which is a foundation base, sunscreen, suntan lotion, milk or cream, artificial suntan lotion, milk or cream, shaving cream or foam, aftershave, shampoo, lipstick, mascara or nail varnish.

40. The topical composition of claim 19, further comprising at least one additional ingredient selected from the group comprising: organic or hydroglycolic solvents, extracted or synthetic fats, ionic or non-ionic thickeners, softeners, opacifiers, stabilizers, silicones, α- or β-hydroxyacids, preservatives, sequestering agents, gelling and viscosifying polymers, surfactants and emulsifiers, plant extracts, tissue extracts, marine extracts, sun filters, moisturizing agents, softening agents, products for the treatment of skin diseases, germicides, propellants, cleaning agents, hair conditioning agents, skin conditioning agents, hair styling agents, hair growth promoters, perfumes, sunscreen and/or sunblock compounds for hair and/or skin, detergents, pharmaceuticals, emollients, antiseptic agents, abrasives, absorbents, aesthetic components such as fragrances, pigments, colorings/colorants for hair and skin, make-up agents, essential oils, skin sensates, astringents, anti-acne agents, anti-caking agents, antifoaming agents, antimicrobial agents, binders, biological additives, buffering agents, bulking agents, chelating agents, chemical additives, cosmetic astringents, cosmetic biocides, denaturants, drug astringents, external analgesics, film formers or materials, opacifying agents, pH adjusters, propellants, reducing agents, sequestrants, skin bleaching and lightening agents, skin-conditioning agents, humectants, skin soothing and/or healing agents, skin treating agents, peptides and peptide derivatives thereof, vitamins and derivatives thereof wetting agents, artificial sun-tanning agents; water-soluble sunscreens; antiperspirants, deodorants, astringents, and refreshing, tonic, cicatrizing, keratolytic and depilatory products, perfumed water, botanical extracts, plant tissue extracts, water-soluble colorants, anti-dandruff agents, antiseborrheic agents, oxidants, reducing agents, vitamins B₁ through B₁₂, C, D, H, K and their derivatives, peptide or steroid hormones, enzymes, coenzymes, enzyme inhibitors, enzyme activators, vaccines, steroidal or non-steroidal anti-inflammatories, antibiotics, antimicrobial and bactericidal substances, cytotoxic or antineoplastic agents, fat-soluble active substances, substances intended to improve the state of dry or aged skin, tocopherols, vitamins E, F or A and their esters, retinoic acid, antioxidants, essential fatty acids, glycyrrhetinic acid, keratolytics and carotenoids, ceramides and pseudo-ceramides, and all lipid complexes of a form similar to that of the natural ceramides of the skin.

41. An article comprising: a synthetic or natural fiber, wool or material useful for the manufacture of clothing or underclothing, wipes, patches, compresses, cottons, cotton buds, dressings, makeup-removal sponges, masks and any other carrier liable to come into direct contact with the skin or scalp coated or impregnated with the topical composition of claim 19.

42. A method of treating, preventing or reducing all outward visibly and tactilely perceptible manifestations of excess weight, skin firming, cellulite or orange peel, comprising the steps of: providing a topical composition as defined in claim 19 and applying same to the skin of a man or woman in need of such treatment.

43. A method of therapeutically regulating excess weight, inducing a slimming treatment of weight overloads of the thighs and the hips, refining contours of the face, reducing the steatomery of the thighs, the volume/fatty overload of the spare tire at the hips, of reducing “love handles,” comprising the steps of: providing a topical composition as defined in claim 19 and applying same to the skin of a man or woman in need of such treatment.
